(19) United States
(12) Patent Application Publication
(10) Pub. No.: US 2001/0037809 A1
(43) Pub. Date: Nov. 8, 2001

(54) Method for the evaluation of air time for scuba divers when diving

(76) Inventor: Giovanni Distefano, Torino (IT)

Correspondence Address: LARSON & TAYLOR, PLC, 1199 NORTH FAIRFAX STREET, SUITE 900, ALEXANDRIA, VA, 22314, US

(21) Appl. No.: 09/847,379

(22) Filed: May 3, 2001

(30) Foreign Application Priority Data
May 5, 2000 (IT) GE2000A000065

(51) Int. Cl. B63C 11/02; G08B 3/00; G08B 5/00; A62B 9/00; A61M 16/00; A62B 27/00; A62B 7/00; F16K 31/02; A62B 7/04; F16K 31/26

(52) U.S. Cl. 128/201.27; 128/204.21; 128/202.22; 128/205.23; 128/204.26

(57) ABSTRACT

Method for the evaluation of the diving air time for a scuba diver equipped with bottles and with a computer for diving, comprising the following steps:

input into said computer of data referring to the initial pressure of the said bottles and to the volume of the said bottles;

acquisition by the said computer of an initial diver's air consumption, said air consumption being an initial default datum;

calculation of an ideal state referring to pressure reduction in the said bottles and therefore of an ideal remaining air time, said calculation being carried out on the basis of a depth datum measured by the said computer and on the basis of the said data introduced in the previous stages;

diver's check on the actual pressure of the bottles at given or arbitrary times;

possible input of the said actual pressure of the bottles into the said computer and calculation of a new state referring to pressure reduction in the bottles and therefore of a new actual remaining air time.

### BACKGROUND AND SUMMARY OF THE INVENTION

The present invention relates to a method for the evaluation of the diving air time for a scuba diver equipped with bottles and with a computer for diving.

Air time, as is generally known, indicates the remaining duration of the air contained in the bottles for a scuba diver who stops at a certain depth and keeps on breathing following a given breathing rhythm. The calculation of said remaining air time is usually carried out by means of the computer for diving, which is also used by the diver in order to get other information concerning, for instance, air pressure in the bottles, the depth she diving at, water temperature and so on. In order to carry out said calculation of air time, the computer has to obtain a certain amount of information, among which bottle pressure, and this is usually done by means of a pressure sensor connected both to the bottle output (for instance on the first reduction stage) and to a data processing unit in the computer. The connection of the sensor to said processing unit is carried out first through means for the transduction/transmission of the data obtained from the bottles, and then through devices converting said data from an analogical form to a digital one, the latter being processable by said computer unit. The use of the pressure sensor proves difficult and expensive, beside increasing the number of devices the diver has to carry during the diving session.

The present invention, therefore, aims at providing a method for the evaluation of air time in which the scuba diver directly communicates to the computer for diving the parameters which are necessary for the calculation of air time, among which the remaining pressure in the bottles, thus avoiding the mounting of a sophisticated and expensive pressure sensor and of corresponding means for the transduction, transmission and conversion of signals to the computer.

Said aim is achieved by the present invention through a method for the evaluation of the diving air time for a scuba diver equipped with bottles and with a computer for diving. Said method includes the following stages:

input into said computer of data about the initial pressure of the bottles and the volume of the bottles;

acquisition by the computer of an initial diver's air consumption; said consumption is an initial default datum or alternatively introduced by the diver, or it refers to previous diving sessions and has therefore been previously stored in the computer;

calculation of an ideal state referring to pressure reduction in the bottles and therefore of an ideal remaining air time, said calculation being carried out on the basis of a depth datum measured by the computer and on the basis of the data introduced in the previous stages;

diver's checking of the actual pressure of the bottles at given or arbitrary times;

possible input of the actual pressure of the bottles into the computer and calculation of a new state referring to pressure reduction in the bottles and therefore of a new actual remaining air time.

Advantageously, according to the method of the present invention, the diver directly operates onto the computer for diving by providing, at given or arbitrary times, the data referring to the instantaneous pressure of the bottles during the diving session, thus allowing said computer for diving to calculate the remaining air time.

### BRIEF DESCRIPTION OF THE DRAWINGS

Further aims and advantages of the present invention will be better understood in the following description, regarded as a mere non-limiting example, with reference to the enclosed drawings, in which:

FIG. 1 schematically shows the equipment necessary to the diver in order to use a method for the evaluation of air time for scuba divers while diving according to the present invention;

FIG. 2 is a block diagram referring to the main stages of the present method for the evaluation of air time; e

FIG. 3 shows a diagram referring to the ideal and actual pressure reduction of the bottles as a function of time.

### DESCRIPTION OF THE PREFERRED EMBODIMENT OF THE INVENTION

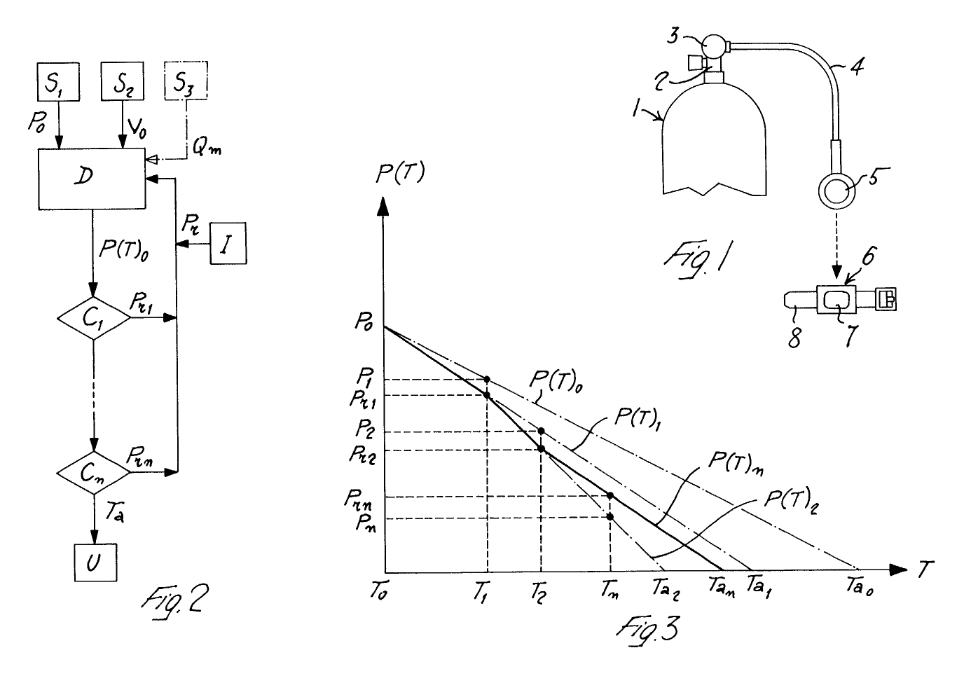

With reference to FIG. 1, the numeral 1 indicates a partially shown bottle, said bottle being pressurized and comprising on its output a distribution valve 2 connected to a first stage 3 of pressure reduction. Said first reduction stage 3 is connected to a flexible pipe or intake 4 acting as a support for a manometer 5, which detects air pressure within the bottle. Beside the bottle 1 and the manometer 5, during a diving session the scuba diver is also equipped with a known computer 6, which comprises in the present form of embodiment a display 7 and which can be tied around the diver's wrist by means of a strap 8. The computer 6 for diving could obviously be positioned otherwise on the diver's equipment and be connected to said equipment through support means other than the strap 8.

By means of the computer 6 the diver can evaluate the remaining air time and can therefore establish the duration of the diving session: see on this the block diagram about the evaluation method shown in FIG. 2. Said diagram comprises the following stages: two stages S₁ and S₂ for the input into the computer 6 of the initial pressure P₀ and of the volume V₀ of the bottle 1 respectively; a stage S₃ for the acquisition by the computer 6 of the diver's initial air consumption Qₘ, said initial pressure P₀ of the bottle 1 being read by means of the manometer 5 and the air consumption Qₘ being set as an arbitrary initial value, as an average value detected by the computer 6 during a previous diving session or as a default value; a stage D for the calculation by said computer 6 of the state P(T) of pressure in the bottle as a function of time T, of the state of air consumption Qₘ (T) in time (not shown), and therefore for the calculation of a remaining air time Ta on the basis of the data input in the stages S₁, S₂ and S₃; a series of arbitrary stages C₁, Cₙ for the diver's check on the actual pressure Pᵣ of the bottles at given times T after the beginning of the diving session through the use of the manometer 5; a possible stage I for the input into the computer 6 by the diver of the actual pressures Pᵣ in the bottle 1 at said times and for the possible recalculation by the computer of the pressure state P(T) of the bottle and of the air consumption Qₘ in time T; eventually, a stage U for the communication by the computer to the scuba diver of the air times Ta and of the remaining pressure P₁ calculated in the bottle 1 at time T; said information can be shown onto the display 7 of the computer 6 or communicated to the diver for instance by radio.

Let's now see the detailed description of the above mentioned stages of the present method, with reference also to FIG. 3, which shows the state of pressure reduction in the bottle 1 as a function of the diving time T. Before diving, the scuba diver must initialize the variables to be input into the computer 6 according to the stages S₁ and S₂, and the computer 6 is switched to a first initializing mode “SET” to this purpose: the acquired variables, as was mentioned, are the initial pressure P₀ of the bottle 1, the volume V₀ of the bottle and the air consumption Qₘ, possibly input by the diver during stage S₃ or directly obtained by the computer 6 as a default value or as a previously stored value. After inputting said variables, the diver switches the computer into a second working mode, the “DIVE” mode, in which the processing unit of said computer calculates, according to stage D, an ideal state P(T)₀ for the pressure reduction as a function of time T and of the depth variations, shown in FIG. 3 with a hatched line whose angular coefficient is a function of the air consumption Qₘ. Said ideal state P(T)₀ finally provides an ideal air time Tao for the diver before the pressure of the bottles reaches a final value considering a certain safety margin, so that said pressure does not sink to zero. The computer also provides at any time T the bottle pressure obtained on the basis of said state P(T)₀: with reference to this, observe on the diagram the pressure P₁ referring to time T₁. According to the diagram in FIG. 2, at a given time T₁ after the beginning of the diving session, the scuba diver carries out a check C₁ on the pressure of the bottle 1 by means of a manometer 5 and reads a certain pressure value Pᵣ₁: if said value Pᵣ₁ differs from the value P₁ shown on the display 7, the diver inputs (stage I) said value Pᵣ₁ into the computer 6, for instance by touching a suitable key which, when pressed, reduces the pressure datum from the value P₁ calculated on the basis of the ideal state P(T)₀ to the read value Pᵣ₁. In the following stage D the computer will carry out a new evaluation and visualization (stage U) of an air time Ta₁ which does not correspond to the ideal value Ta₀ and which is obtained on the basis of a new pressure state P(T)₁. Said air time Ta₁ at the diver's disposal is thus evaluated with respect to the already past time T₁ and it is adapted to the new value of air consumption Qₘ calculated by the computer 6 considering the pressure correction carried out at stage I. After the check C₁ and after establishing the new air time Ta₁, the diver carries out a new check at time T₂, inputting at stage I into the computer 6 the new pressure Pᵣ₂ read from the manometer 5, and will then obtain, in the same way as before, a new air time Ta₂ on the basis of another state P(T)₂. Said air time Ta₂ will obviously consider the already past time T₂. As described for the checks at diving times T₁ and T₂, the diver can arbitrarily carry out other pressure checks onto the bottle; note in particular from FIG. 3 that at time Tₙ the pressure of bottle 1 has a value Pᵣ₁ which is obviously lower than the previous values Pᵣ₁ and Pᵣ₂, but it is higher than the pressure Pₙ obtained on the basis of the previous state P(T)₂: this means that the state P(T)ₙ decreases with a lower inclination with respect to the states P(T)₁ and P(T)₂, then the computer 6, on the basis of the pressure correction Pᵣₙ input by the diver, has recalculated an air consumption Qₘ lower than the situations referring to times T₁ and T₂; this can be due for in-stance to a slower breathing rhythm. Finally, it should be noted that the display 7 of the computer, beside air time and bottle pressure at a given time, can show other data useful to the diver, such as reached depth, decompression times, water temperature and others.

1. Method for the evaluation of the diving air time for a scuba diver equipped with bottles and with a computer for diving, comprising the following steps:
input into said computer of data referring to the initial pressure of the said bottles and to the volume of the said bottles;
acquisition by the said computer of an initial diver's air consumption, said air consumption being an initial default datum;
calculation of an ideal state referring to pressure reduction in the said bottles and therefore of an ideal remaining air time, said calculation being carried out on the basis of a depth datum measured by the said computer and on the basis of the said data introduced in the previous stages;
diver's check on the actual pressure of the bottles at given or arbitrary times;
possible input of the said actual pressure of the bottles into the said computer and calculation of a new state referring to pressure reduction in the bottles and therefore of a new actual remaining air time.

2. Method according to claim 1 in which the said actual remaining air time is obtained from the said computer during the stage of calculation on the basis of an actual air consumption calculated as a function of the actual pressure of the bottles.

3. Method according to claim 1, in which the said computer is provided with a display showing the said remaining air time, the pressure of the said bottles and other information which can be useful to the diver.

4. Method according to claim 2, in which during the said stage of initial input the said computer takes an average air consumption value detected and stored on the basis of calculation carried out during a previous diving session of the diver.

5. Method according to claim 1, in which the said computer includes a key to decrease and/or in-crease the pressure value of the bottles from an initial value calculated at a given value pre-established by the diver.

6. Method according to claim 1, in which the said computer is provided with an initializing mode for the data referring to the said initial pressure of the said bottles and to the volume of the said bottles.

7. Method according to claim 1, in which the data referring to the pressure of the said bottles are read by the diver by means of a manometer connected to said bottles.
